(19) United States
(12) Patent Application Publication
(10) Pub. No.: US 2024/0041859 A1
(43) Pub. Date: Feb. 8, 2024

(54) METHODS OF TREATING AND PREVENTING KIDNEY DISEASE

(71) Applicant: Washington University, St. Louis, MO (US)

(72) Inventors: Mohamed 'Moe' Mahjoub, St. Louis, MO (US); Tao Cheng, St. Louis, MO (US)

(73) Assignee: Washington University, St. Louis, MO (US)

(21) Appl. No.: 18/255,655

(22) PCT Filed: Dec. 2, 2021
(86) PCT No.: PCT/US2021/061586

§ 371 Date: Jun. 2, 2023

Related U.S. Application Data
(60) Provisional application No. 63/120,951, filed on Dec. 3, 2020.

(51) Int. Cl. A61K 31/4745; A61P 13/12; A61K 31/473

(52) U.S. Cl. CPC A61K 31/4745; A61P 13/12; A61K 31/473

(57) ABSTRACT

Among the various aspects of the present disclosure is the provision of a method of treating or preventing kidney disease in a subject in need thereof. Another aspect of the present disclosure provides for a method of reducing a number of kidney cells having excess centrosomes or centrosome amplification (CA) or inhibiting centrosome clustering in a subject having or suspected of having a kidney disease associated with CA or increased centrosome clustering. In some embodiments, the method comprises administering to the subject a centrosome clustering inhibiting agent.

### CROSS-REFERENCE TO RELATED APPLICATIONS

This application claims priority from U.S. Provisional Application Ser. No. 63/120,951 filed on Dec. 3, 2020, which is incorporated herein by reference in its entirety.

### STATEMENT REGARDING FEDERALLY SPONSORED RESEARCH OR DEVELOPMENT

This invention was made with government support under DK108005 awarded by the National Institutes of Health and W81XWH-20-1-0198 awarded by the U.S. Army Medical Research and Materiel Command (ARMY/MRMC). The government has certain rights in the invention.

### MATERIAL INCORPORATED-BY-REFERENCE

Not applicable.

### FIELD OF THE INVENTION

The present disclosure generally relates to prevention or treatment of a kidney disease.

### SUMMARY OF THE INVENTION

Among the various aspects of the present disclosure is the provision of a method of treating or preventing kidney disease in a subject in need thereof comprising administering to the subject a therapeutically effective amount of a centrosome clustering inhibiting agent. Another aspect of the present disclosure provides for a method of reducing a number of kidney cells having excess centrosomes or centrosome amplification (CA) in a subject having or suspected of having a kidney disease associated with CA or increased centrosome clustering comprising administering to the subject an amount of a centrosome clustering inhibiting agent effective to inhibit centrosome clustering or reduce an amount of kidney cells having CA. Yet another aspect of the present disclosure provides for a method of inhibiting centrosome clustering in a subject having or suspected of having a kidney disease associated with centrosome amplification (CA) or increased centrosome clustering comprising administering to the subject an amount of a centrosome clustering inhibiting agent effective to inhibit centrosome clustering or reduce an amount of cells having CA. In some embodiments, the subject has an increased number of kidney cells having more than one centrosome or centrosome amplification (CA) compared to a healthy subject. In some embodiments, the subject has a kidney disease associated with centrosome amplification (CA), excess centrosomes, or centrosome clustering.

In some embodiments, the kidney cells having centrosome amplification (CA) have ectopic centrosomes. In some embodiments, the subject has or is suspected of having a ciliopathy, a disease caused by ciliary dysfunction polycystic kidney disease. In some embodiments, the subject has or is suspected of having polycystic kidney disease. In some embodiments, the subject does not have cancer. In some embodiments, the centrosome clustering inhibiting agent is a poly(ADP-ribose) polymerase 1 (PARP1) inhibiting agent. In some embodiments, the centrosome clustering inhibiting agent is a CPAP-tubulin interaction inhibiting agent. In some embodiments, the centrosome clustering inhibiting agent is PJ-34 or CCB02. In some embodiments, administering the centrosome inhibiting agent to a subject having or kidney cells with centrosome amplification (CA) results in increased formation of multipolar mitoses, increases activation of SAC, and causes mitotic catastrophe and cell death, which reduces the number of kidney cells having excess centrosomes. In some embodiments, the centrosome clustering inhibiting agent targets kidney cells having abnormal or amplified centrosomes. In some embodiments, the amount of the centrosome clustering inhibiting agent is an amount sufficient to reduce, inhibit, or block centrosome amplification (CA), aggregation, or clustering in a kidney or kidney cells, resulting in negative selection and cell death of kidney cells with more than one centrosome. In some embodiments, the amount of the centrosome clustering inhibiting agent is an amount sufficient to reduce or eliminate a fraction of kidney cells having centrosome amplification (CA) in a kidney cell population while not impacting normal mitotic spindle formation in kidney cells with normal centrosome number. In some embodiments, the amount of the centrosome clustering inhibiting agent is an amount sufficient to promote multipolar spindle configuration or formation of multipolar mitoses in centrosome amplification (CA) kidney cells, resulting in mitotic catastrophe and cell death or activate a spindle assembly checkpoint (SAC), leading to CA kidney cell death. In some embodiments, the amount or amount of the centrosome clustering inhibiting agent is an amount sufficient to reduce or inhibit defective asymmetric cell division, defective cilia assembly, or secretion of proliferation-driving cytokines in a kidney or kidney cells. In some embodiments, the amount of the centrosome clustering inhibiting agent is an amount sufficient to decrease or reduce renal fibrosis or scarring. In some embodiments, the amount or therapeutically effective amount of the centrosome clustering inhibiting agent is an amount sufficient to reduce or decrease cyst initiation, formation, or growth in kidneys or kidney cells, reduce cystogenesis in kidneys or kidney cells, or decrease cyst number, reduce cystic burden, and reduce overall fractional cyst area in kidneys. In some embodiments, the amount or therapeutically effective amount of the centrosome clustering inhibiting agent is an amount sufficient to improve or retain kidney filtration function, inhibit or block an increase in serum creatinine (Cr) and blood urea nitrogen (BUN), or retain or improve renal morphology and function. In some embodiments, the amount or therapeutically effective amount of the centrosome clustering inhibiting agent is an amount sufficient to reduce kidney size or reduce renal damage. In some embodiments, the amount or therapeutically effective amount of the centrosome clustering inhibiting agent is an amount sufficient to increase reduction of cyst formation compared to tolvaptan. In some embodiments, reducing centrosome amplification in a kidney or kidney cells, results in a decrease or inhibition of cell-autonomous and non-cell-autonomous defects that contribute to, and accelerate, a cystic disease phenotype. In some embodiments, the amount or therapeutically effective amount of the centrosome clustering inhibiting agent is administered to the subject about daily or about every two days for about sixty days or about five months or more, and, optionally, re-administering the centrosome clustering inhibiting agent upon a recurrence of kidney disease. In some embodiments, the amount or therapeutically effective amount of the centrosome clustering inhibiting agent is administered orally. In some embodiments, the amount or therapeutically effective amount of the centrosome clustering inhibiting agent is administered to the subject before the subject exhibits a decline in kidney function or increase in kidney size. In some embodiments, administering the amount or therapeutically effective amount of the centrosome clustering inhibiting agent prevents or reduces cyst formation, fibrosis, or an increase in serum creatinine or BUN for at least three months after administration.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a schematic showing how centrosome clustering helps cells with centrosome amplification survive mitotic catastrophe and cell death.

FIG. 2A-FIG. 2C. Centrosome amplification (CA) is evident in autosomal dominant polycystic kidney disease (ADPKD) cell lines. (A) Immunofluorescence staining of control and pkd1-null cells with markers of centrosomes. Arrows indicate cells with CA in pkd1-mutant cells. (B, C) Quantification of the extent of centrosome amplification in pdk1-mutant cells.

FIG. 3A-FIG. 3D. Centrosome-clustering inhibitors block centrosome aggregation and force the formation of multipolar spindle configuration in ADPKD cell lines. (A, B) Example images of wild-type (HK2) and pkd1-null (WT9-12) cells upon treatment with centrosome clustering inhibitors. (C, D) Treatment of cells with either PJ-34 or CCB02 inhibited centrosome clustering in cells with CA, while not impacting normal mitotic spindle formation in cells with normal centrosome number.

FIG. 4. Experimental outline for testing of centrosome clustering inhibitors during the accelerated cystogenesis stage in the slow-onset ADPKD mouse model (top) and graph of weight loss for mice treated with centrosome amplification inhibitors (bottom).

FIG. 5A-FIG. 5B. Inhibition of centrosome clustering for two months promotes multipolar spindle configurations in vivo. (A) Images of kidney cells from mice treated with CCB02 or PJ-34, showing spindle configurations. (B) Quantification of the fraction of cells showing multipolar and pseudo-bipolar spindle configurations.

FIG. 6A-FIG. 6C. Inhibition of centrosome clustering for two months reduces the cystic burden in ADPKD kidneys. (A) Example images of kidneys of PKD1^{RC/RC} mice treated with either inhibitor, showing significant reduction in cyst formation. Treatment with either inhibitor reduces the kidney size (B), cyst number, and overall fractional cyst area (C).

FIG. 7A-FIG. 7B. Inhibition of centrosome clustering for two months reduces renal damage and improves kidney filtration function. (A) Example images of kidneys stained by trichrome or anti-SMA to highlight fibrotic cells. (B) Quantification of the kidney area covered by fibrosis, serum creatinine, and BUN.

FIG. 8. Mice were administered vehicle-only (control), CCB02, or tolvaptan (gavage every other day for 5 months). Experimental outline for testing of centrosome clustering inhibitors at the onset of kidney function decline in a slow-onset ADPKD mouse model.

FIG. 9A-FIG. 9B. Inhibition of centrosome clustering for an extended period of five months results in a greater reduction of cystogenesis in ADPKD kidneys. (A) Example images of kidneys of PKD1^{RC/RC} treated with either inhibitor, showing significant reduction in cyst formation. Treatment with either inhibitor reduces the kidney size, cyst number, and overall fractional cyst area (B).

FIG. 10A-FIG. 10C. Inhibition of centrosome clustering for five months significantly reduces renal fibrosis. (A, B) Example images of kidneys of PKD1^{RC/RC} treated with either CCB02 or tolvaptan, showing significant reduction in fibrosis markers quantified in (C).

FIG. 11. Inhibition of centrosome clustering for five months maintains kidney filtration function. Treatment with either inhibitor blocks the increase in serum creatinine and BUN, indicating rescue of kidney function.

FIG. 12A-FIG. 12D. ADPKD mice demonstrate long-term improvement in kidney morphology and function following treatment with centrosome clustering inhibitors. (A) Experimental design. (B) Compared to untreated mice, PKD1^{RC/RC} administered CCB02 showed a reduction in cyst formation at 11 months. Analysis of kidneys at 3 months post-washout indicate that cysts do not reform, that fibrosis is also not evident (C), and there is no increase in serum creatinine or BUN (D).

### DETAILED DESCRIPTION OF THE INVENTION

The present disclosure is based, at least in part, on the discovery that centrosome amplification in developing embryonic kidneys can cause cystic kidney disease. As shown herein, blocking the proliferation of these cells with excess centrosomes—by inhibiting centrosome clustering—ameliorated the cystic kidney disease phenotype.

Human renal disorder or polycystic kidney disease is caused by the over-proliferation of kidney epithelial cells, which causes the development of cysts in the nephron tubules. These cysts grow until they impede kidney filtration and function, and result in end-stage renal failure. Here, the objective was to identify cellular pathways that one can target to stop the proliferation of these cells, and thus improve kidney morphology and retain kidney function.

Recently, a defect in the biogenesis of centrosomes in kidney cells that can drive cyst growth was identified. Centrosomes are essential microtubule-based organelles that are key to cell division. Described here is the testing of small molecule inhibitors that can target these cells with abnormal centrosomes, and two compounds that act to inhibit a process termed “centrosome clustering”.

One of these compounds (PJ-34, a PARP inhibitor) is commercially available but has not been used in this context (e.g., polycystic kidney disease).

The other compound (CCB02, an inhibitor of CPAP-tubulin interaction) was generated for use in the context of cancer. Here, the molecule is being studied for use in mouse models of polycystic kidney disease.

Described herein is the use of the commercially available inhibitor, PJ-34, and CCB02 for use in the treatment and prevention of kidney diseases associated with abnormal centrosomes, such as polycystic kidney disease. These results have confirmed that targeting “centrosome clustering” is an effective treatment for kidney disease, such as polycystic kidney disease.

Centrosome Clustering Inhibiting Agent

One aspect of the present disclosure provides for targeting centrosome clustering, proteins associated with centrosome clustering, or downstream signals to treat kidney disease. The present disclosure provides methods of treating or preventing kidney dysfunction or disease based on the discovery that a defect in the biogenesis of centrosomes in kidney cells drives cyst growth. Any centrosome clustering inhibiting (CCi) agents identified in the context of treating cancer can be used herein (e.g., Sabat-Pospiech et al. Biochem Soc Trans (2019) 47 (5): 1209-1222; Pannu et al. Centrosome-declustering drugs mediate a two-pronged attack on interphase and mitosis in supercentrosomal cancer cells. Cell Death Dis 5, e1538 (2014); Kawamura et al. Oncotarget. 2013; 4:1763-1776). Centrosome amplification (CA) which leads to centrosome clustering, can be targeted by inhibition of centrosome inactivation, inhibition of centrosome-dependent invasion, or by reversing CA itself, thereby reversing the pathogenic phenotypes associated with CA.

As described herein, inhibitors of centrosome clustering (e.g., small molecules) can reduce or prevent cyst formation in kidneys. A centrosome clustering inhibiting agent can be any agent that can inhibit centrosome clustering.

As an example, a centrosome clustering inhibiting agent can inhibit a PARP or CPAP-tubulin interaction. For example, the centrosome clustering inhibiting agent can be PJ-34 or CCB02.

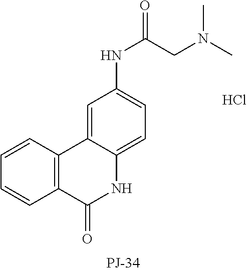 [SMILES: CN(C)CC(=O)Nc1ccc2[nH]c(=O)c3ccccc3c2c1 ; Cl]

PJ-34 is a potent inhibitor of poly(ADP-ribose) polymerase (PARP), an enzyme involved in DNA repair and cell proliferation, that dose-dependently inhibits purified PARP enzyme in a cell-free assay with half-maximal effective concentration EC₅₀ value of 20 nM. Unlike other PARP inhibitors (such as 3-AB), PJ-34 does not possess any antioxidant properties but exhibits 10,000 times greater PARP inhibition than 3-AB (EC₅₀=200 μM).

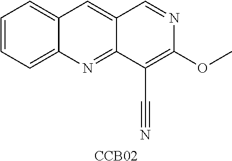 [SMILES: COc1ncc2cc3ccccc3nc2c1C#N ; "CCB02"]

CCB02 is a selective CPAP-tubulin interaction inhibitor, binding to tubulin and competing for the CPAP binding site of P-tubulin, with an IC50 of 689 nM, and shows potent anti-tumor activity. Screening for compounds that perturb CPAP-tubulin interaction led to the identification of CCB02, which selectively binds at the CPAP binding site of tubulin. Genetic and chemical perturbation of CPAP-tubulin interaction activates extra centrosomes to nucleate enhanced numbers of microtubules prior to mitosis.

It is believed that any compound that blocks centrosome clustering could be used for polycystic kidney disease. That is, the selected compounds were chosen (PJ-34 and CCB02) because they target different proteins. This was to show that targeting centrosome clustering itself is the key step, not the specific protein being targeting per se. As such, other centrosome clustering inhibiting (CCi) agents such as GF-15, griseofulvin, CP-673451, RedBr-Nos, crenolanib, CW069, AZ82 (data not shown), SR31527, AZ0108 can also be useful for treating kidney disease.

Kidney Diseases, Disorders, or Conditions

Compositions and methods described herein can treat a kidney disease, disorder, or condition associated with centrosome clustering (e.g., ciliopathy, kidney disease, such as with a cystic disease phenotype or polycystic kidney disease (e.g., ADPKD, PKD).

Ciliopathy

Kidney diseases characterized by dysregulation of epithelial cell physiology, cytoskeletal morphology, and hyperproliferation of normally quiescent tubular cells (e.g., autosomal dominant polycystic kidney disease (ADPKD)) profoundly alter the organ architecture and impair renal function. It is now established that ADPKD is a “ciliopathy,” or a disease caused by ciliary dysfunction. Cilia are microtubule-based structures that play important chemo- and mechanosensory roles in cells. In the kidney, cilia are found on renal progenitor cells during embryonic development and epithelial cells lining the various segments of mature nephrons. The cilia protrude from the apical surface and are in contact with the extracellular environment, acting as a cellular sensor that regulates epithelial cell growth, homeostasis, and repair. The assembly of cilia is templated by the centrosome, the major microtubule-organizing center in mammalian cells. Most cells in the human body, including the kidney, contain a solitary centrosome and cilium, and cells have evolved tight regulatory mechanisms to ensure each cell contains only one of each organelle.

Centrosome Amplification (CA)

Kidney diseases, disorders, or conditions treatable by the disclosed compositions and methods can be associated with centrosome amplification (CA).

Centrosome amplification (CA) is the formation of too many centrosomes in a cell. It causes multiple cellular defects including (a) mitotic chromosome segregation errors and genome instability, (b) defective asymmetric cell division and fate determination, (c) invasive cell behavior leading to metastasis, (d) defective cilia assembly and cell-cell-signaling, and (e) secretion of cytokines that can drive the proliferation of neighboring cells. All of these defects have been described in the context of cancer. However, since CA can disrupt ciliary function, one major question was whether CA is also observed in ciliopathies (and not just cancer) (see e.g., Nigg and Holland, Nature Reviews—Mol. Cell Bio (2018).

Previous work has shown centrosome amplification is prevalent in kidneys of patients with autosomal dominant polycystic kidney disease (ADPKD) (see e.g., Dionne et al. (2018) JCB). Human ADPKD samples were stained for markers of centrosomes and the extent of CA in normal tubules, small cysts, and large cysts were quantified. Centrosome amplification occurs early during cyst formation and persists as cysts grow and enlarge. But, it remained unclear whether this defect was causing the disease phenotype, or simply correlated with cystogenesis.

The previous work also showed that in mouse models where centrosome amplification can be induced in embryonic kidneys in vivo, centrosome amplification causes cystic kidney disease shortly after birth. Kidneys were isolated from control (WT) and mice expressing Plk4 to induce CA in the renal collecting duct system. This was the first example showing that centrosome amplification can cause a cystic kidney disease phenotype. Although overall cell proliferation decreased as kidney development finished postnatally, cells with CA showed enhanced proliferation at stages when they should become quiescent. Here, how these cells survive the mitotic catastrophe and proliferate is examined. The mechanism of “centrosome clustering” may help these cells avoid apoptosis.

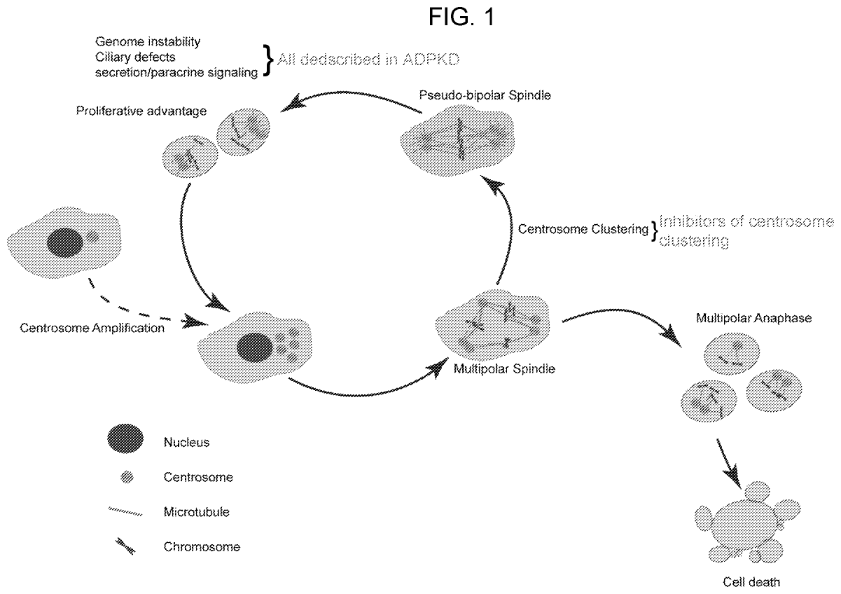

Centrosome amplification is normally detrimental to cell survival. Cells undergo multipolar spindle configurations in mitosis, resulting in gross aneuploidy and negative selection. But, “centrosome clustering” is a survival response that cells adapt to avoid this mitotic catastrophe and cell death (see e.g., FIG. 1). It was found that kidney cells with too many centrosomes also adapt this centrosome clustering mechanism to avoid cell death. Genome instability, ciliary defects, and secretion/paracrine signaling are all described in ADPKD. Thus, inhibiting centrosome clustering may be a strategy to force these cells with too many centrosomes into negative selection and cell death.

Recent studies of cystic epithelia in human and mouse ADPKD tissues have identified a phenomenon termed centrosome amplification (CA; meaning more than one centrosome per cell). CA is most commonly caused by deregulated centrosome biogenesis and is detrimental to cell physiology in three main ways: (1) it causes abnormal mitotic spindle formation, leading to chromosome missegregation errors and genome instability; (2) it disrupts cilia assembly and signaling; and (3) it results in increased secretion of cytokines and pro-inflammatory paracrine signaling. Collectively, these changes enhance the proliferation of cells and tissues with CA.

The inventors recently demonstrated that CA is prevalent in cystic kidneys of ADPKD patients and that induction of CA in developing mouse kidneys can promote cystogenesis in vivo. In addition to the cell-intrinsic defects (genome instability and aberrant ciliogenesis), these cells also secrete cytokines and cause pro-inflammatory paracrine signaling. In essence, they act as “signal amplifiers,” promoting the proliferation of adjacent cells. Thus, it was hypothesized that targeting and killing cells with CA (more than one centrosome per cell) will attenuate cystic epithelial growth and improve renal morphology and function.

Amplified centrosomes normally cause multipolar mitoses, resulting in mitotic catastrophe and cell death. However, cells with CA can adopt a pseudo-bipolar spindle configuration via a process termed “centrosome clustering,” a key mechanism by which cells circumvent mitotic catastrophe and apoptosis. Thus, inhibiting centrosome clustering can be a strategy to counteract the proliferation of cells with high incidences of centrosome amplification. To facilitate this, various drug discovery screens identified selective small molecule inhibitors of centrosome clustering (e.g., CCB02 and PJ-34).

This phenomenon has not been well studied in kidney disease, as such the inventors are believed to be the first to investigate this in-depth. There is much more focus on this phenomenon in cancer.

Autosomal Dominant Polycystic Kidney Disease (ADPKD)

Autosomal dominant polycystic kidney disease (ADPKD) is considered one of the most common life-threatening inherited genetic diseases. The incidence of ADPKD in the population is high, affecting up to 1 in 400 births across all racial and ethnic groups, impacting −13 million individuals worldwide. ADPKD is a significant cause of kidney failure, accounting for −5% of patients with end-stage renal disease (ESRD) requiring renal transplant and dialysis, at an annual cost of more than $1.5 billion in the United States alone. Almost half of the patients develop kidney failure by the fifth or sixth decades of life, in conjunction with extrarenal manifestations such as hypertension, abdominal pain, and polycystic liver disease. Management of ADPKD is disproportionately costly, as approximately 6%-10% of all individuals receiving dialysis and renal transplant in the United States are afflicted with ADPKD. Yet, the current state of therapy for ADPKD is dismal. Despite multiple attempts with trials of targeted therapeutics, none has been an unmitigated success in the clinic. The only therapeutic approved in the United States, tolvaptan, reduces the rate of decline in renal function by a mere 2%-3% annually. Moreover, there remain concerns regarding liver toxicity and increased urine volume, which can result in considerable interference with the quality of life. Thus, the nephrology community is left with renal dialysis and transplantation as the only viable option. None of these treatments are satisfactory for such a common disease in the 21st century, and new therapeutic directions are desperately needed.

Another aspect of the present disclosure provides for methods of assessing, preventing, and treating other renal pathologies or improving renal function in a subject having or suspected of having a renal pathology, disease, disorder, or condition. For example, a renal pathology, disease, or disorder can be Alagille syndrome, Alport syndrome, amyloidosis, chronic kidney disease (CKD), cystinosis, diabetic neuropathy (DN), end-stage renal disease (ESRD), Fabry disease, focal segmental glomerulosclerosis, glomerulonephritis, Goodpasture syndrome, atypical hemolytic uremic syndrome (aHUS), hemolytic uremic syndrome (HUS), Henoch-Schonlein purpura, hypertensive kidney disease, IgA nephropathy (Berger's disease), interstitial nephritis, kidney cancer, lupus nephritis, minimal change disease, nephropenia, nephrotic syndrome, polycystic kidney disease (PKD), renal cell carcinoma, renal sarcoma, renal vascular disease, thrombotic thrombocytopenic purpura (TTP), granulomatosis with polyangiitis (GPA), transitional cell carcinoma, urothelial cell carcinoma, or Wilms tumor.

Formulation

The agents and compositions described herein can be formulated in any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating, preventing, or reversing a kidney disease, disorder, or condition associated with centrosome clustering (e.g., ciliopathy, kidney disease, such as with a cystic disease phenotype or polycystic kidney disease (ADPKD, PKD) in a subject in need of administration of a therapeutically effective amount of a centrosome clustering inhibiting agent, so as to reduce or inhibit cells with centrosome amplification, cyst formation, cell-autonomous or non-cell-autonomous defects, or interstitial fibrosis and improve renal function.

As described in Example 1, using ADPKD human and mouse experimental data, it was recently discovered that abnormal biogenesis of centrosomes, resulting in an increase in their number in tubular cells, plays a crucial role in cyst development and growth. Essentially, these cells behave as “bad actors” that impact not only their progeny during cell proliferation, but also non-dividing neighboring tubular cells to drive cyst formation. Based on these findings, it was hypothesized that targeting and eliminating these cells will reduce cystogenesis and improve renal function in ADPKD patients. Here is shown, strong evidence demonstrating that cystic cells with ectopic centrosomes, the cells whose proliferation leads to ADPKD, can be selectively killed when treated with novel compounds that specifically target cells with abnormal centrosomes. The elimination of these cells results in improvements in kidney morphology and function, highlighting the feasibility of this approach. This disclosure focuses on the preclinical evaluation of two such small molecule inhibitors, CCB02 and PJ-34, in blocking cyst initiation and growth in ADPKD mice and cultured cells. These inhibitors were tested using slow-onset mouse models of ADPKD. These inhibitors may attenuate cystogenesis in rapid-onset kidney disease or following acute kidney injury, which is known to accelerate the cystic disease phenotype and progression to renal failure.

Methods described herein are generally performed on a subject in need thereof. A subject in need of the therapeutic methods described herein can be a subject having, diagnosed with, suspected of having, or at risk for developing a kidney disease, disorder, or condition. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a centrosome clustering inhibiting agent is, for example, an amount that would cause the desired therapeutic effect in a subject while minimizing undesired side effects. In various embodiments, an effective amount of a centrosome clustering inhibiting agent described herein can substantially inhibit or reduce centrosome clustering, slow the progress of a kidney disease, disorder, or condition, or limit the development of a kidney disease, disorder, or condition.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a centrosome clustering inhibiting agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit or reduce centrosome clustering; slow the progress of a kidney disease, disorder, or condition; limit the development of a kidney disease, disorder, or condition; reduce or inhibit centrosome amplification, cyst formation, cell-autonomous and non-cell-autonomous defects, or interstitial fibrosis; or improve renal function.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a centrosome clustering inhibiting agent can occur as a single event or over a time course of treatment. For example, a centrosome clustering inhibiting agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for a kidney disease, disorder, or condition associated with centrosome clustering.

A centrosome clustering inhibiting agent can be administered simultaneously or sequentially with another agent, such as a second centrosome clustering inhibiting agent, standard kidney disease treatment (e.g., tolvaptan), an antibiotic, an anti-inflammatory, or another agent. For example, a centrosome clustering inhibiting agent can be administered simultaneously with another agent, such as a second centrosome clustering inhibiting agent, standard kidney disease treatment (e.g., tolvaptan), an antibiotic, or an anti-inflammatory. Simultaneous administration can occur through administration of separate compositions, each containing one or more of a centrosome clustering inhibiting agent, a second centrosome clustering inhibiting agent, standard kidney disease treatment (e.g., tolvaptan), an antibiotic, an anti-inflammatory, or another agent. Simultaneous administration can occur through administration of one composition containing two or more of a centrosome clustering inhibiting agent, a second centrosome clustering inhibiting agent, standard kidney disease treatment (e.g., tolvaptan), an antibiotic, an anti-inflammatory, or another agent. A centrosome clustering inhibiting agent can be administered sequentially with a second centrosome clustering inhibiting agent, standard kidney disease treatment (e.g., tolvaptan), an antibiotic, an anti-inflammatory, or another agent. For example, a centrosome clustering inhibiting agent can be administered before or after administration of a second centrosome clustering inhibiting agent, standard kidney disease treatment (e.g., tolvaptan), an antibiotic, an anti-inflammatory, or another agent. For example, combination treatment can be CCB02+Tolvaptan (data not shown) and PJ-34+Tolvaptan. These combination treatments can be toxic, thus titering and testing various concentrations of each compound when used together can be performed.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Screening

Also provided are methods for screening for a centrosome clustering inhibiting agent using ADPKD cell lines (e.g., cystic epithelial cells isolated from patients with PKD) or mouse models, such as slow-onset mouse model of PKD, called the Pkd1^{RC/RC} mice, or mice where centrosome amplification is induced in embryonic kidney cells. It was shown here that this can indeed cause cystic kidney disease. This was the first example showing that centrosome amplification can cause a cystic kidney disease phenotype.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Inhibition of Centrosome-Clustering Reduces Cystogenesis and Improves Kidney Function in Autosomal Dominant Polycystic Kidney Disease (ADPKD)

Described herein are experiments that tested whether pharmacological inhibition of centrosome clustering can attenuate cystic cell growth and expansion in ADPKD mice. Here, two newly characterized small molecule inhibitors of centrosome clustering, CCB02 and PJ-34, were tested using slow-onset models of ADPKD. Here, inventors characterize the effects of centrosome clustering inhibitors on cyst initiation and growth in vivo. The data with these compounds provide a new rationale for the clinical evaluation of such inhibitors in patients with ADPKD.

This example describes how centrosome amplification in the kidney causes cell-autonomous and non-cell-autonomous defects that contribute to, and accelerate, the cystic disease phenotype in ADPKD. This suggests that targeting cells with excess centrosomes may provide a therapeutic avenue.

This example also describes how inhibition of centrosome clustering in ADPKD cells with excess centrosomes in vitro promotes the formation of multipolar mitoses, activation of the spindle assembly checkpoint (SAC), and leads to mitotic catastrophe and cell death. These cells are thus eliminated from the remaining population.

This example also describes how inhibition of centrosome clustering in ADPKD mice in vivo during the “accelerated cystogenesis” stage results in decreased cyst formation, reduced interstitial fibrosis, and improved renal filtration function. Moreover, treatment of ADPKD mice earlier (at the onset of cyst formation) and for an extended period showed even more dramatic improvements in kidney morphology and function.

This example also describes how treatment of ADPKD mice, followed by washout of the centrosome clustering inhibitors, resulted in less cystic kidneys—suggesting there are long-term benefits to removing the cells with excess centrosomes from ADPKD kidneys.

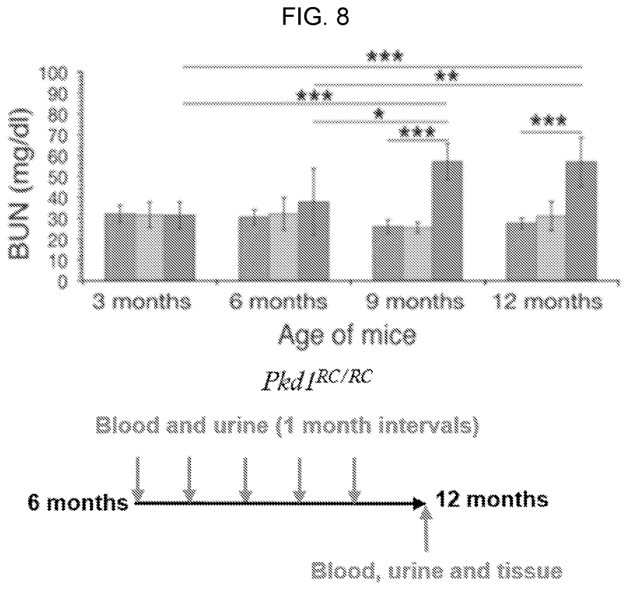

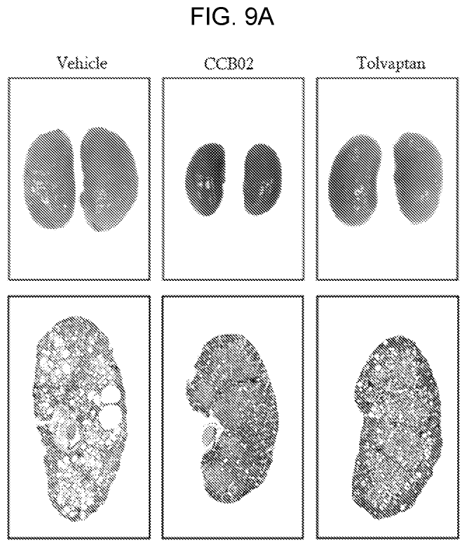

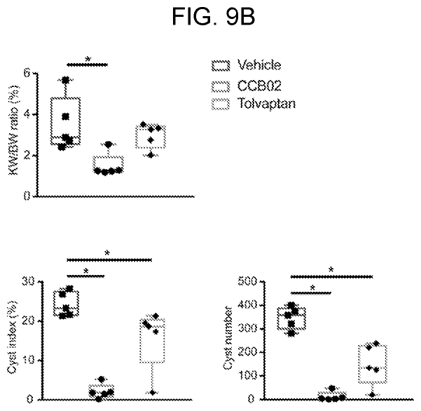

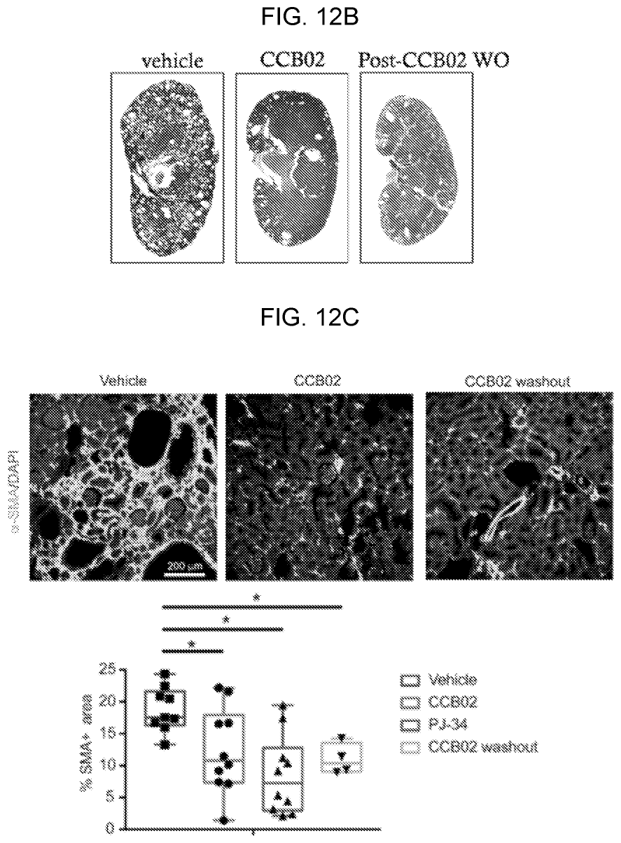

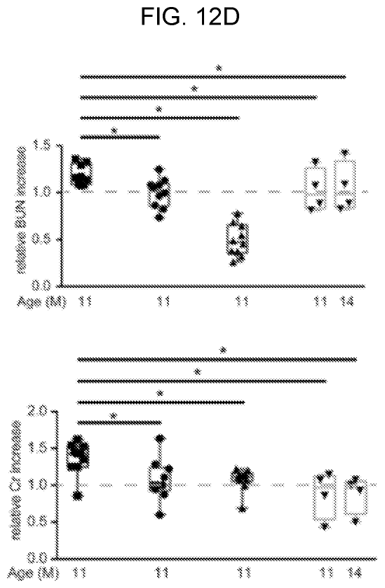

This example further describes experiments performed that show prevention of disease in the mouse model from reaching a stage when their kidneys were very cystic and failing. FIG. 8-FIG. 9 shows the results from an experiment where the mice are treated very early when cystogenesis has not fully progressed. There is nearly an 80% reduction in cyst formation in these animals. These results also show long-term benefits of treatment with the centrosome clustering inhibitor: even after treatment with the centrosome clustering inhibitors has been stopped, the cyst growth does not recur (see e.g., FIG. 12).

In summary, this example describes the feasibility of using inhibitors of centrosome clustering as a preventative and therapeutic strategy in polycystic kidney disease.

Centrosome Clustering Inhibitors (CCi)

There have been several high-throughput screens to identify inhibitors of centrosome clustering. However, the vast majority have only been tested in vitro. Different inhibitors were selected to test in mouse models of autosomal dominant polycystic kidney disease (ADPKD). The criteria for inclusion were inhibitors must target different proteins with divergent mechanisms of action with regards to centrosome clustering, inhibitors were screened against cells with normal centrosome number, and the inhibitors were tested in mice, indicating that mice can tolerate the drug. Two such inhibitors, PJ-34 (PARP inhibitor) and CCB02 (inhibits CPAP-tubulin interaction), met the inclusion criteria and have been previously tested in mice and shown to be effective in blocking centrosome clustering. Thus, these two inhibitors were used to block clustering in subsequent experiments in ADPKD mice and cells.

Characterization of CCis on ADPKD Cell Lines

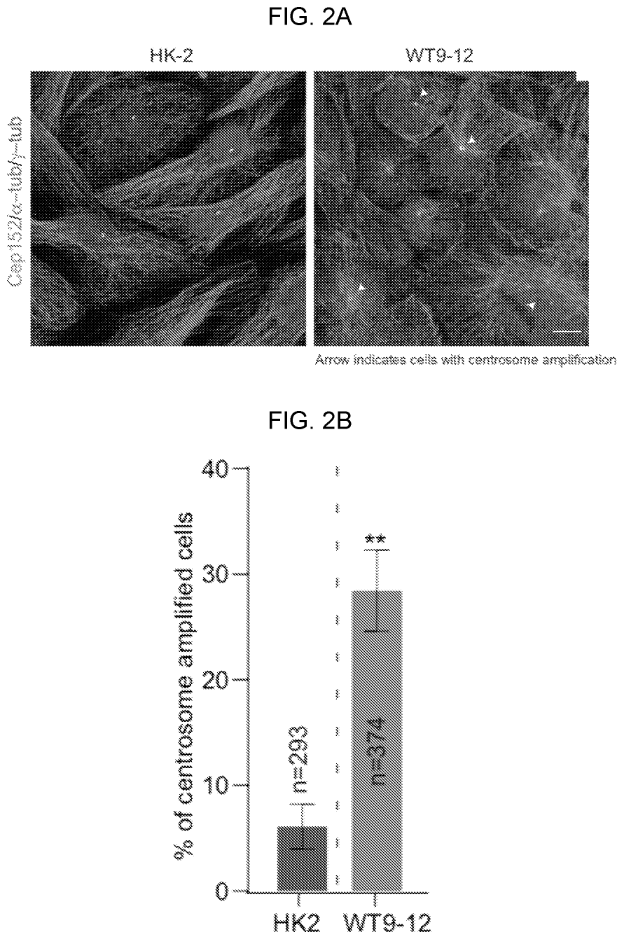

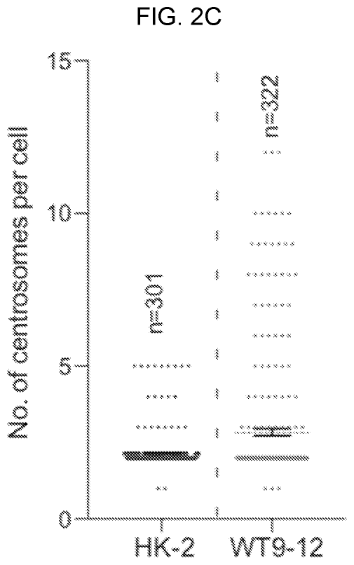

First, it was observed that centrosome amplification is evident in ADPKD cell lines, highlighting their utility as an in vitro model to test the centrosome clustering inhibitors (see e.g., FIG. 2A-FIG. 2C). Unlike wild-type cells, pkd1-mutant cells displayed significant levels of centrosome amplification, similar to what was observed in kidney tissues of ADPKD patients.

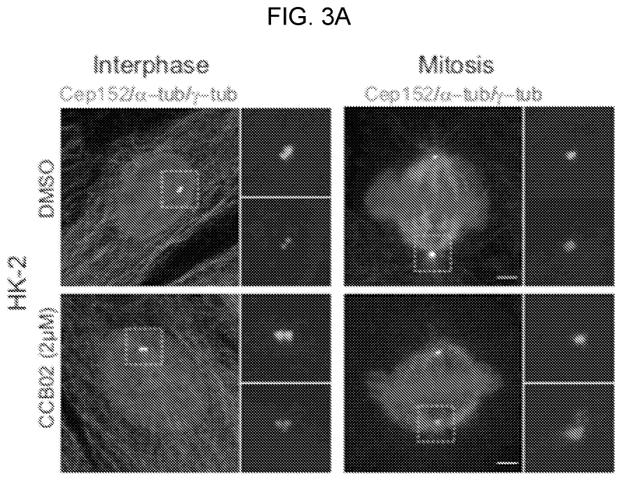

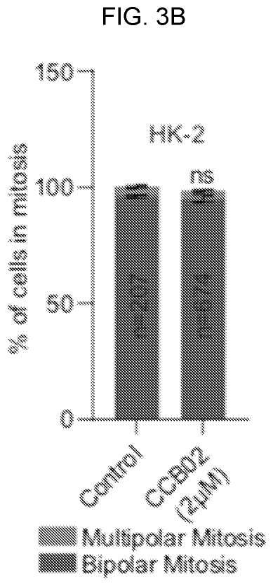

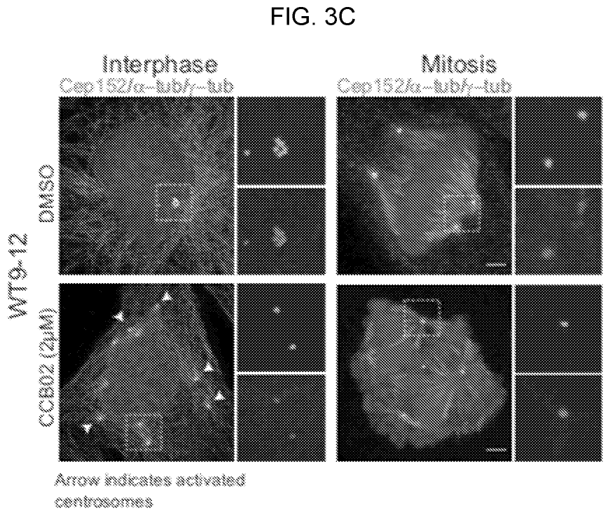

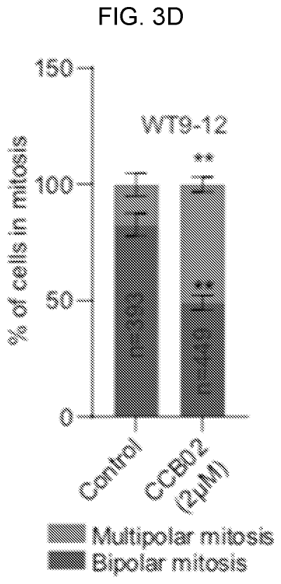

Centrosome-clustering inhibitors (CCis) were shown to block centrosome aggregation and force the formation of multipolar spindle configuration in ADPKD cell lines (see e.g., FIG. 3A-FIG. 3D). Wild-type (HK2) and pkd1-null (WT9-12) cells were treated with either PJ-34 or CCB02, which inhibited centrosome clustering in cells with CA as predicted, while not impacting normal mitotic spindle formation in cells with normal centrosome number (see e.g., FIG. 3A-FIG. 3D). This caused a delay in mitotic progression (see e.g., FIG. 3C and FIG. 3D), and the activation of the spindle assembly checkpoint (SAC), leading to cell death and elimination of cells with CA from the population. Since this approach worked in ADPKD cells, mouse models were subsequently used to test the efficacy of the inhibitors in vivo.

ADPKD Mouse Models

To test the inhibitors, a slow-onset mouse model of ADPKD was needed that would allow for an extended period of time to treat these mice with the centrosome clustering inhibitors. The widely used slow-onset ADPKD model called the PKD1^{RC/RC} mouse (Hopp et al, JCI, 2012) was chosen for the small molecule inhibitor tests. These mice develop cysts slowly, such that kidney size increases around 6 months of age, and kidney function declines—as indicated by elevated blood-urea nitrogen (BUN) levels—around 9 months of age. The mice also show extensive cystogenesis as they age. Importantly, quantification of centrosome number showed a similar range in the fraction of cells with CA, consistent with what was observed in ADPKD patient tissues. Thus, this model was ideal for testing the centrosome clustering inhibitors. Using the slow-onset ADPKD model, the optimal effective time-course for inhibition of centrosome clustering using CCB02 and PJ-34 can be determined. Example 1 describes treatment at timepoints (e.g., beginning at six months of age, beginning at nine months, and up to eleven months of age) and for periods of time (e.g., every other day for sixty days or two months, every two days for five months).

ADPKD Mouse Models Treated with CCis

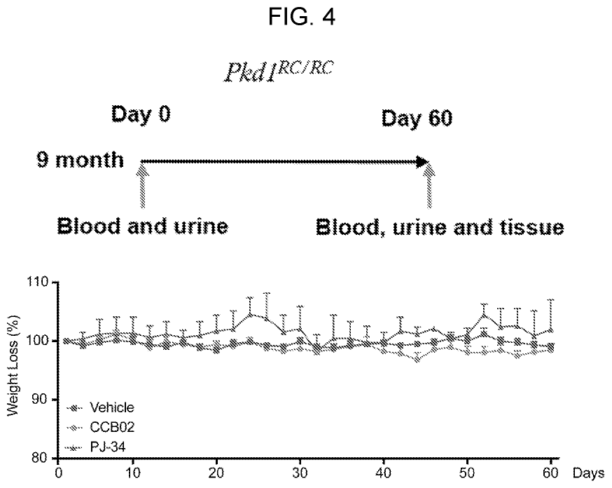

First, PKD1^{RC/RC} mice were treated with the centrosome clustering inhibitors beginning at nine months of age—at a stage when their kidneys were beginning to rapidly become cystic (see e.g., FIG. 4). Each compound was administered by gavage every two days for a total of 60 days. Quantification of animal weight and feeding behavior indicated that the mice tolerated both compounds very well, feeding habits were normal, and the mice maintained their weight throughout the treatment regimen (see e.g., FIG. 4).

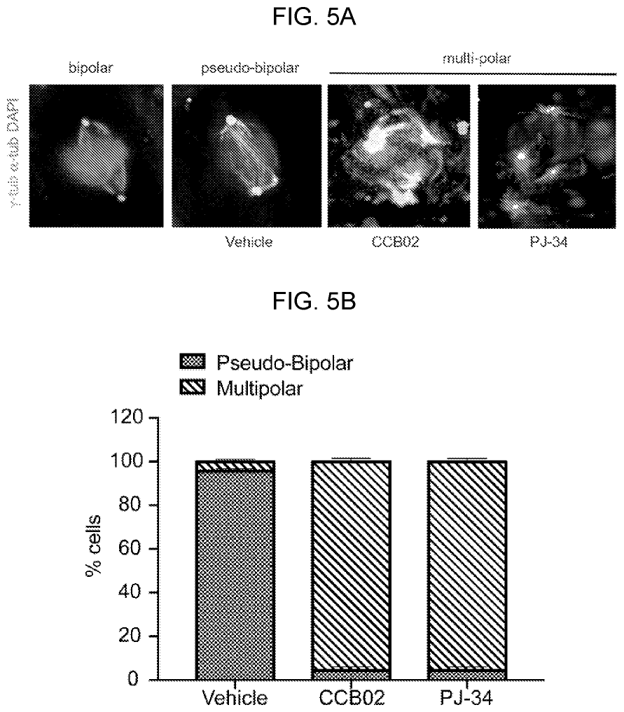

Examination of kidneys isolated from mice treated with the centrosome clustering inhibitors for two months indicated that these compounds were effective at blocking centrosome clustering in vivo, similar to what was observed in vitro (see e.g., FIG. 5A-FIG. 5B). Quantification of the fraction of cells with CA demonstrated that the majority of the cells failed to cluster their centrosomes, and form multipolar mitotic spindle configurations. Thus, these compounds had the desired effect on kidney tissues and cells.

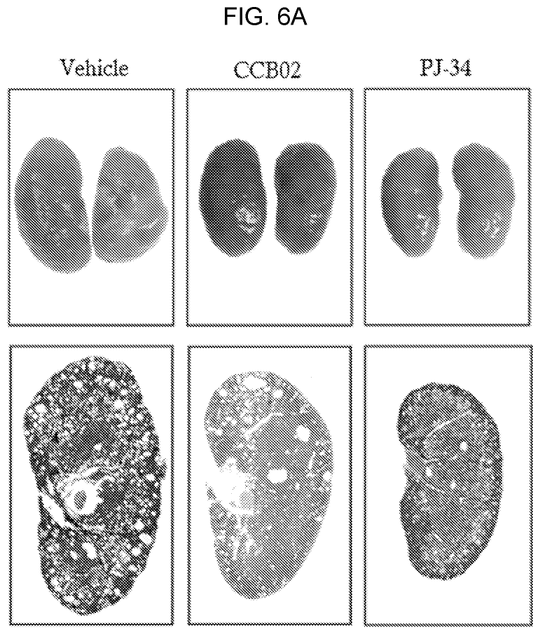

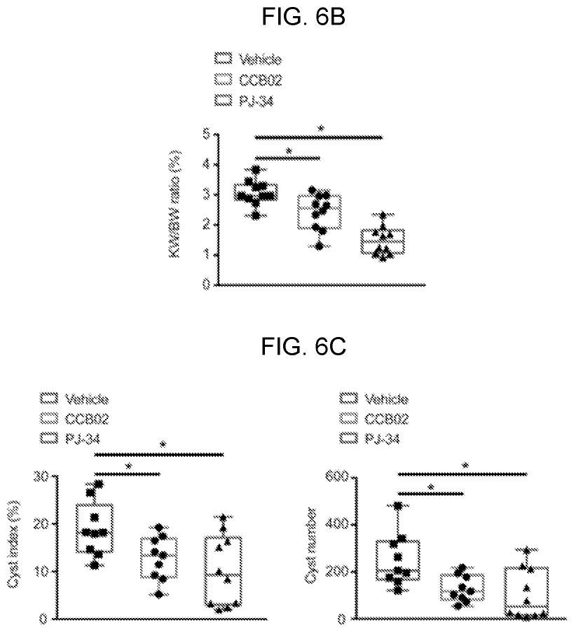

Treatment of mice for two months with either inhibitor showed a great effect on reducing cystogenesis and helped retain kidney function (see e.g., FIG. 6A-FIG. 6C). Images of kidneys of PKD1^{RC/RC} mice treated with either inhibitor showed a significant reduction in cyst formation (see e.g., FIG. 6A). Treatment with either inhibitor reduced the kidney size, cyst number, and overall fractional cyst area (see e.g., FIG. 6B and FIG. 6C).

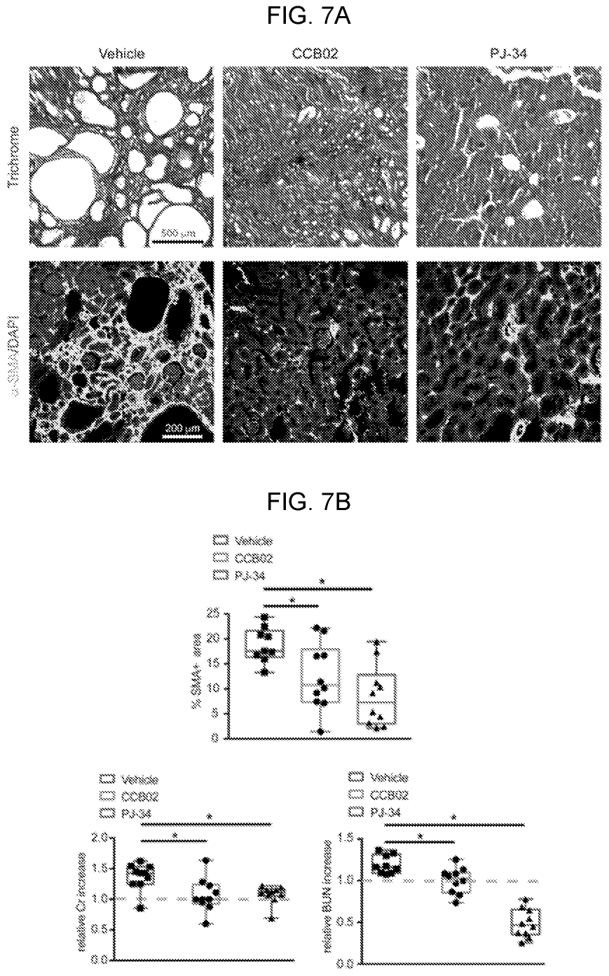

Treatment of mice for two months with either inhibitor also showed a significant effect on kidney fibrosis, which is caused by cyst growth, damages the renal interstitium, and disrupts kidney function (see e.g., FIG. 7A-FIG. 7B). Treatment with either inhibitor reduced the kidney area covered by fibrosis and the increase in serum creatinine and BUN, indicating a “rescue” of kidney function (see e.g., FIG. 7B). Based on the success of this short treatment window, inhibitors were subsequently tested in mice for a longer period of time.

PKD1^{RC/RC} mice were treated with the inhibitors beginning at six months of age—at a stage when their kidneys have yet to dramatically increase in size and show functional decline (see e.g., FIG. 8). Centrosome clustering inhibitor CCB02 was administered by gavage every two days for a total of five months. Also included in the analysis was the only FDA-approved drug for ADPKD, tolvaptan. This is a Vasopressin V2-receptor antagonist, so it has a completely different mechanism of action (e.g., does not inhibit centrosome clustering), and serves as an additional control. Blood and urine were collected at one-month intervals to monitor kidney function (see e.g., FIG. 8).

Treatment of mice for five months with CCB02 showed an even greater effect on cystogenesis compared to the two-month treatment scheme (see e.g., FIG. 9A and FIG. 9B). Kidneys of PKD1^{RC/RC} mice treated with either inhibitor showed a significant reduction in cyst formation (see e.g., FIG. 9A). Treatment with either inhibitor also reduced the kidney size, cyst number, and overall fractional cyst area (see e.g., FIG. 9B). Importantly, the centrosome clustering inhibitor, CCB02, yielded better results in comparison to the FDA-approved drug tolvaptan in reducing cyst formation.

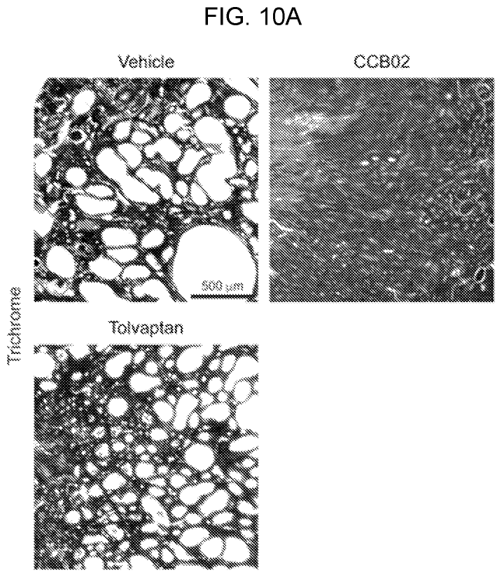

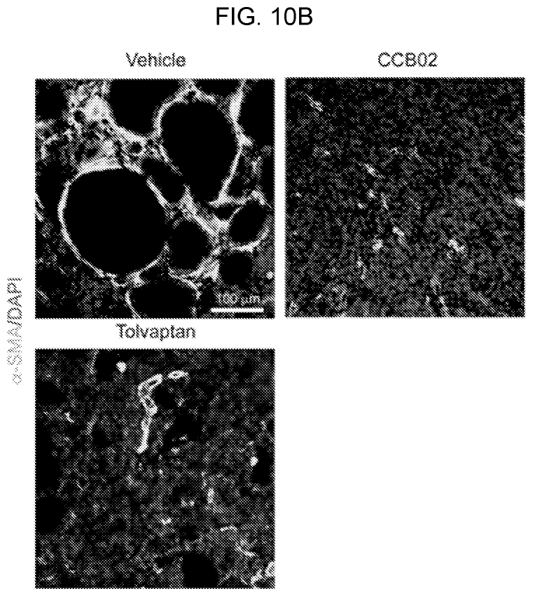

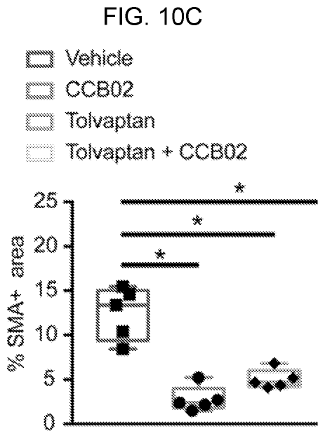

Kidneys of PKD1^{RC/RC} mice treated with either CCB02 or tolvaptan for five months showed a significant reduction in fibrosis markers (see e.g., FIG. 10A-FIG. 10C). Treatment of mice for five months with CCB02 showed an even greater effect on renal fibrosis compared to the two-month treatment scheme.

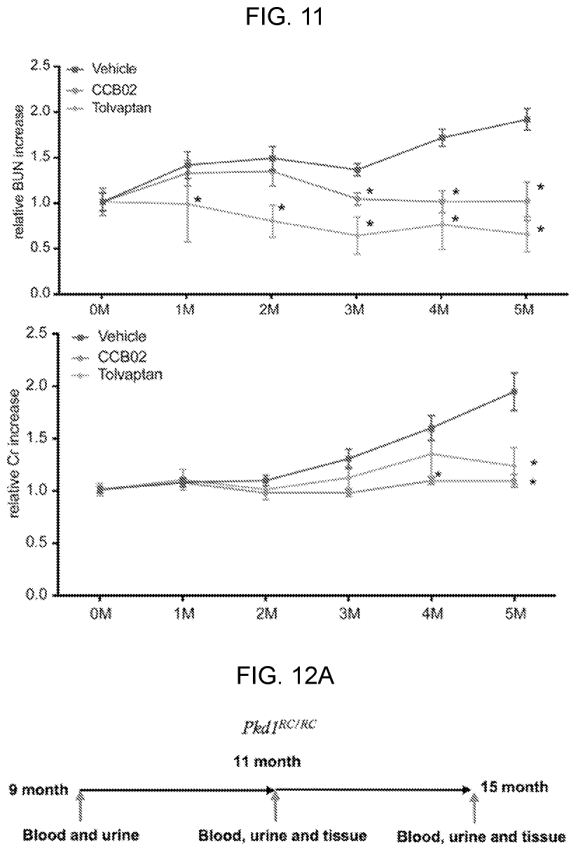

Inhibition of centrosome clustering for five months also maintained kidney filtration function. Treatment with either inhibitor blocked the increase in serum creatinine and BUN, indicating rescue of kidney function (see e.g., FIG. 11). The levels of creatinine and BUN did not significantly increase relative to the starting level (baseline), whereas vehicle-treated mice showed gradual elevation of these markers indicative of kidney filtration failure.

ADPKD mice also demonstrated long-term improvement in kidney morphology and function following treatment with centrosome clustering inhibitors (see e.g., FIG. 12A-FIG. 12D). PKD1^{RC/RC} mice were administered centrosome clustering inhibitor CCB02 beginning at nine months and up to eleven months of age (see e.g., FIG. 12A). CCB02 was administered by gavage every other day for sixty days (see e.g., FIG. 8). The treatment was arrested for three months, and animals were monitored for three additional months to determine if cysts returned once the compounds are no longer administered. Compared to untreated mice, PKD1^{RC/RC} mice administered CCB02 showed a reduction in cyst formation at eleven months (see e.g., FIG. 12B). Analysis of kidneys at three months post-washout indicated that cysts did not reform, that fibrosis was not evident, and there was no increase in serum creatinine or BUN (see e.g., FIG. 12C and FIG. 12D). Collectively, these data point to potential long-term benefits of using centrosome clustering inhibitors in ADPKD.

### SUMMARY

In summary, centrosome amplification in the kidney causes cell-autonomous and non-cell-autonomous defects that contribute to, and accelerate, the cystic disease phenotype in ADPKD. This suggests that targeting cells with excess centrosomes may provide a therapeutic avenue. As shown herein, in vitro inhibition of centrosome clustering in ADPKD cells with excess centrosomes promotes the formation of multipolar mitoses, activation of the SAC, and leads to mitotic catastrophe and cell death. Those cells are then eliminated from the remaining population.

As shown herein, in vivo inhibition of centrosome clustering in ADPKD mice during the accelerated cystogenesis stage results in decreased cyst formation, reduced interstitial fibrosis, and improved renal filtration function. Moreover, treatment of ADPKD mice earlier (at the onset of cyst formation) and for an extended period showed even more dramatic improvements in kidney morphology and function.

Treatment of ADPKD mice, followed by washout of the centrosome clustering inhibitors, results in fewer cystic kidneys, suggesting there are long-term benefits to removing the cells with excess centrosomes from the kidneys of ADPKD mice.

The mechanisms by which inhibiting centrosome clustering attenuates cystogenesis can be identified by identifying intra- and extracellular signaling pathways attenuated by centrosome clustering inhibitors in vitro, using human and mouse PKD cells and characterizing signaling pathways affected by CA and attenuated by centrosome clustering inhibitors in vivo, using ADPKD and centrosome amplification-inducible mouse models.

Overall, the data shown herein demonstrates the feasibility of using centrosome clustering inhibitors as a new therapeutic strategy in ADPKD.

1-28. (canceled)

29. A method of treating or preventing kidney disease in a subject in need thereof comprising: administering to the subject a centrosome clustering inhibiting agent.

30. The method of claim 29, wherein the subject has or is suspected of having a polycystic kidney disease.

31. The method of claim 30, wherein the polycystic kidney disease is a ciliary dysfunction polycystic kidney disease.

32. The method of claim 29, wherein the subject does not have cancer.

33. The method of claim 29, wherein the centrosome clustering inhibiting agent is a poly(ADP-ribose) polymerase 1 (PARP1) inhibiting agent.

34. The method of claim 29, wherein the centrosome clustering inhibiting agent is a CPAP-tubulin interaction inhibiting agent.

35. The method of claim 29, wherein the centrosome clustering inhibiting agent is PJ-34 or CCB02.

36. The method of claim 29, wherein the centrosome clustering inhibiting agent is administered orally.

37. A method of reducing kidney cell centrosome amplification (CA) in a subject in need thereof, the method comprising:
administering to the subject a centrosome clustering inhibiting agent effective to reduce a number of kidney cells having CA.

38. The method of claim 37, wherein the subject has an increased number of kidney cells having CA compared to a healthy subject.

39. The method of claim 37, wherein the number of kidney cells having CA comprise ectopic centrosomes.

40. The method of claim 37, wherein the centrosome clustering inhibiting agent is a poly(ADP-ribose) polymerase 1 (PARP1) inhibiting agent.

41. The method of claim 37, wherein the centrosome clustering inhibiting agent is a CPAP-tubulin interaction inhibiting agent.

42. The method of claim 37, wherein the centrosome clustering inhibiting agent is PJ-34 or CCB02.

43. A method of inhibiting kidney cell centrosome clustering in a subject in need thereof, the method comprising:
administering to the subject a centrosome clustering inhibiting agent effective to inhibit kidney cell centrosome clustering.

44. The method of claim 43, wherein administering the centrosome clustering inhibiting agent results in cell death of kidney cells having more than one centrosome.

45. The method of claim 43, wherein administering the centrosome clustering inhibiting agent does not impact normal mitotic spindle formation in kidney cells with a single centrosome.

46. The method of claim 43, wherein the centrosome clustering inhibiting agent is a poly(ADP-ribose) polymerase 1 (PARP1) inhibiting agent.

47. The method of claim 43, wherein the centrosome clustering inhibiting agent is a CPAP-tubulin interaction inhibiting agent.

48. The method of claim 43, wherein the centrosome clustering inhibiting agent is PJ-34 or CCB02.
